(19) United States
(12) Patent Application Publication
(10) Pub. No.: US 2023/0018150 A1
(43) Pub. Date: Jan. 19, 2023

(54) ULTRASONIC ENDOSCOPE AND BALLOON

(71) Applicant: FUJIFILM Corporation, Tokyo (JP)

(72) Inventors: Nori MIYAGISHIMA, Kanagawa (JP); Yasuhiko MORIMOTO, Kanagawa (JP)

(73) Assignee: FUJIFILM Corporation, Tokyo (JP)

(21) Appl. No.: 17/839,456

(22) Filed: Jun. 13, 2022

(30) Foreign Application Priority Data
Jul. 16, 2021 (JP) 2021-117865

(51) Int. Cl. A61B 8/12; A61B 1/00; A61B 8/00

(52) U.S. Cl. CPC A61B 8/12; A61B 1/00073; A61B 1/00082; A61B 1/00101; A61B 8/445

(57) ABSTRACT

Provided are ultrasonic endoscope and a balloon with improved balloon detachment resistance.

An ultrasonic endoscope includes an ultrasound oscillator unit disposed in a distal end part of an insertion part, and a balloon attachably and detachably attached to the distal end part to cover an outside surface of the ultrasound oscillator unit, the distal end part is provided on at least one of a distal end side or a proximal end side of the ultrasound oscillator unit and has balloon attachment grooves formed in an outer peripheral surface of the distal end part along a peripheral direction, the balloon has a balloon body provided with openings, and a first annular locking part and a second annular locking part that are provided in the opening of the balloon body and are locked in the balloon attachment grooves, and the first annular locking part and the second annular locking part have a detachment prevention shape for restraining detachment from the balloon attachment grooves.

### CROSS-REFERENCE TO RELATED APPLICATION

The present application claims priority under 35 U.S.C §119(a) to Japanese Patent Application No. 2021-117865 filed on Jul. 16, 2021, which is hereby expressly incorporated by reference, in its entirety, into the present application.

### BACKGROUND OF THE INVENTION

### 1. Field of the Invention

The present invention relates to an ultrasonic endoscope and a balloon, and in particular, to an ultrasonic endoscope including a balloon mounted on a distal end part of an insertion part, and a balloon.

### 2. Description of the Related Art

In a medical field, an ultrasonic endoscope is used. The ultrasonic endoscope disposes an imaging element and an ultrasound transducer integrally in a distal end part of an insertion part that is inserted into a body cavity of a subject. The ultrasound transducer emits an ultrasonic wave toward a site to be observed in the body cavity and receives an echo signal reflected from the site to be observed, and an electric signal depending on the received echo signal is output to an ultrasonic observation device. Then, the electric signal is subjected to various kinds of signal processing in the ultrasonic observation device, and is then displayed as an ultrasound tomographic image on a monitor or the like.

The ultrasonic wave and the echo signal are considerably attenuated in the air. For this reason, there is a need to interpose an ultrasonic wave transmission medium, such as water or oil, between the ultrasound transducer and the site to be observed. Accordingly, an expandable balloon is mounted on a distal end part of an ultrasonic endoscope, and the ultrasonic wave transmission medium is injected into the balloon to expand the balloon to be brought into contact with the site to be observed. With this, air is eliminated from a region between the ultrasound transducer and the site to be observed, and the attenuation of the ultrasonic wave and the echo signal is restrained.

An example of the ultrasonic endoscope that mounts the balloon in the distal end part of the insertion part and uses the balloon is WO2020/044905A.

### SUMMARY OF THE INVENTION

The balloon is mounted on the insertion part of the ultrasonic endoscope by locking a locking part provided in an end portion of the balloon in an attachment groove provided in the insertion part. Since the insertion part of the endoscope is used in a state of being inserted into the subject, the balloon mounted on the insertion part is required to be resistant to leakage of a fluid from the end portion of the balloon (fluid leakage resistance) and detachment of the balloon during use (balloon detachment resistance). An increase in volume of the locking part of the balloon and improvement of the contractile force of the locking part allow the balloon to be firmly fixed to the insertion part, and result in the improvement of the fluid leakage resistance. Note that, in a case where the volume of the locking part increases, a portion of the locking part protruding from attachment groove is made large. For this reason, in inserting or extracting the insertion part into or from the subject, the locking part may be brought into contact with a wall portion in the subject, the balloon may be likely to be detached, and the balloon detachment resistance may be degraded.

In this way, in the ultrasonic endoscope of the related art, one technical issue is to improve the balloon detachment resistance.

The present invention has been accomplished in view of such a situation, and an object of the present invention is to provide an ultrasonic endoscope and a balloon with improved balloon detachment resistance.

To attain the object of the present invention, there is provided an ultrasonic endoscope according to the present invention comprising an ultrasound oscillator unit disposed in a distal end part of an insertion part, and a balloon attachably and detachably attached to the distal end part to cover an outside surface of the ultrasound oscillator unit, in which the distal end part is provided on at least one of a distal end side or a proximal end side of the ultrasound oscillator unit and has a balloon attachment groove formed in an outer peripheral surface of the distal end part along a peripheral direction, the balloon has a balloon body that has at least one end provided with an opening, and an annular locking part that is provided in the opening of the balloon body and is locked in the balloon attachment groove, and the annular locking part has a detachment prevention shape for restraining detachment from the balloon attachment groove.

According to an aspect of the present invention, it is preferable that, in a cross-sectional shape in a case where the annular locking part is cut in an axial direction perpendicular to a peripheral direction of the annular locking part, the annular locking part has, as the detachment prevention shape, a shape in which a thickness on a balloon body side is thickest and the thickness is thinned toward an end portion on an opposite side to the balloon body.

According to an aspect of the present invention, it is preferable that the annular locking part has a toric part that is connected to the balloon body and is positioned on an outside surface of the distal end part, and a toric protrusion part that protrudes in an inward direction from the toric part and is fitted into the balloon attachment groove, in a cross-sectional shape in a case where the annular locking part is cut in an axial direction perpendicular to a peripheral direction of the annular locking part, in a case where the axial direction is set as a width direction, the toric part is formed to be wider than the toric protrusion part, and the detachment prevention shape includes the toric part and the toric protrusion part.

According to an aspect of the present invention, it is preferable that, a relationship of a height of the toric protrusion part greater than a height of the toric part is satisfied in a case where a direction perpendicular to the axial direction is set as a height direction in the cross-sectional shape of the annular locking part.

According to an aspect of the present invention, it is preferable that the toric part has an internal extension part that extends from a protruding position of the toric protrusion part toward a balloon body side on the outer peripheral surface of the distal end part.

According to an aspect of the present invention, it is preferable that, in a case where the axial direction is set as the width direction in the cross-sectional shape of the annular locking part, and in a case where a width of the toric protrusion part is C, and a width of the internal extension part of the toric part is D₁, C/2 ≤ D₁ ≤ 2C is satisfied.

According to an aspect of the present invention, it is preferable that the toric part has an external extension part that extends from a protruding position of the toric protrusion part toward an opposite side to a balloon body side on the outer peripheral surface of the distal end part.

According to an aspect of the present invention, it is preferable that, in a case where the axial direction is set as the width direction in the cross-sectional shape of the annular locking part, and in a case where a width of the toric protrusion part is C, and a width of the external extension part of the toric part is D₂, C/2 ≤ D₂ ≤ 2C is satisfied.

According to an aspect of the present invention, it is preferable that, in a cross-sectional shape in a case where the annular locking part is cut in an axial direction perpendicular to a peripheral direction of the annular locking part, in a case where the axial direction is set as a width direction, the annular locking part has, as the detachment prevention shape, a fitting shape part in which an inside in a radial direction perpendicular to an axial direction is formed to be wider than an outside, and the balloon attachment groove has a groove shape in which the fitting shape part is fitted into the groove in the width direction with no gap.

According to an aspect of the present invention, it is preferable that the fitting shape part has a shape that is widened to a balloon body side from the outside toward the inside in the radial direction.

According to an aspect of the present invention, it is preferable that the annular locking part is formed separately from the balloon body and is integrated with the balloon body.

According to an aspect of the present invention, it is preferable that the annular locking parts are bonded to the balloon body by adhesion or welding.

According to an aspect of the present invention, it is preferable that the annular locking part is disposed on an inner surface side of the balloon body.

According to an aspect of the present invention, it is preferable that a material of the balloon body is silicone.

According to an aspect of the present invention, it is preferable that the distal end part has, as the balloon attachment groove, a first balloon attachment groove provided on the proximal end side of the ultrasound oscillator unit, and a second balloon attachment groove provided on the distal end side of the ultrasound oscillator unit, and the balloon has, as the annular locking part, a first annular locking part that is locked in the first balloon attachment groove, and a second annular locking part that is locked in the second balloon attachment groove.

To attain the object of the present invention, there is provided a balloon according to the present invention that is used in an ultrasonic endoscope including an ultrasound oscillator unit disposed in a distal end part of an insertion part and having a balloon attachment groove that is provided on at least one of a distal end side or a proximal end side of the ultrasound oscillator unit and formed in an outer peripheral surface of the distal end part along a peripheral direction, and is attachably and detachably attached to the distal end part to cover an outside surface of the ultrasound oscillator unit, the balloon comprising a balloon body that has at least one end provided with an opening, and an annular locking part that is provided in the opening of the balloon body and is locked in the balloon attachment groove, in which the annular locking part has a detachment prevention shape for restraining detachment from the balloon attachment groove.

According to the present invention, it is possible to improve balloon detachment resistance.

### BRIEF DESCRIPTION OF THE DRAWINGS

FIG. 1 is a schematic configuration diagram showing an example of the configuration of an ultrasonography system using an ultrasonic endoscope.

FIG. 2 is a partial enlarged perspective view showing the appearance of an example of a distal end part of the ultrasonic endoscope shown in FIG. 1.

FIG. 3 is a schematic view (side sectional view) of a distal end part of an ultrasonic endoscope of a first embodiment.

FIG. 4 is a schematic view (side sectional view) of a distal end part of an ultrasonic endoscope of a second embodiment.

FIG. 5 is a schematic view (side sectional view) of a distal end part of an ultrasonic endoscope of a third embodiment.

FIG. 6 is a partial enlarged perspective view showing an appearance of an example of a distal end part of an ultrasonic endoscope of a fourth embodiment.

FIG. 7 is a schematic view (side sectional view) of the distal end part of the ultrasonic endoscope of the fourth embodiment.

FIG. 8 is a diagram illustrating a manufacturing method of a balloon.

FIG. 9 is a diagram illustrating another manufacturing method of a balloon.

### DESCRIPTION OF THE PREFERRED EMBODIMENTS

Hereinafter, a preferred embodiment of an ultrasonic endoscope according to the present invention will be described referring to the accompanying drawings.

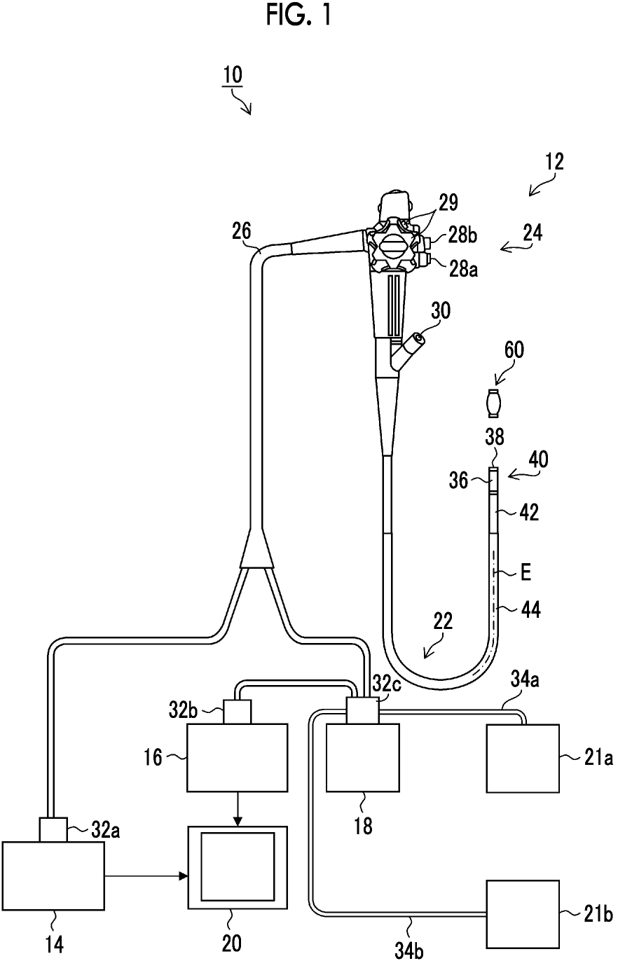

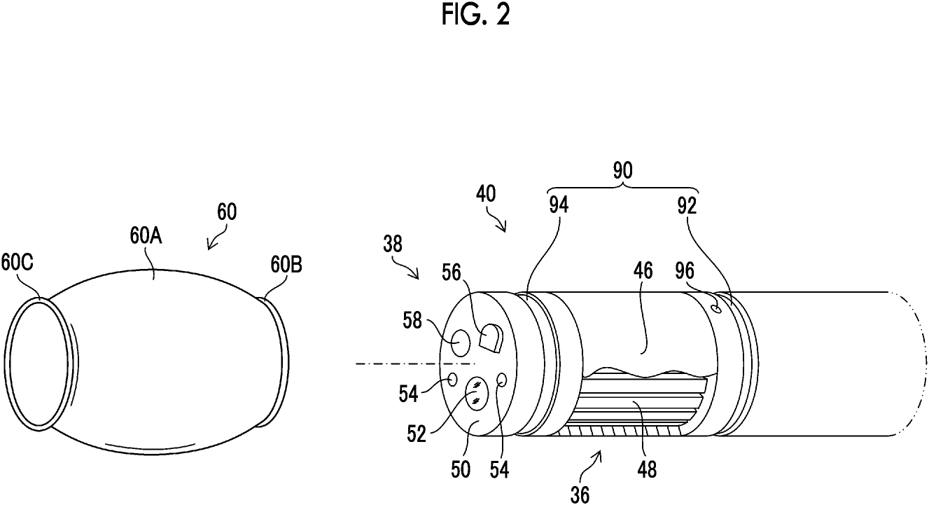

FIG. 1 is a schematic configuration diagram showing an example of an ultrasonography system 10 that uses an ultrasonic endoscope 12 of an embodiment. FIG. 2 is a partial enlarged perspective view showing the appearance of an example of a distal end part of the ultrasonic endoscope shown in FIG. 1.

As shown in FIG. 1, the ultrasonography system 10 comprises the ultrasonic endoscope 12, an ultrasound processor device 14 that generates an ultrasound image, an endoscope processor device 16 that generates an endoscope image, a light source device 18 that supplies illumination light, with which the inside of a body cavity is illuminated, to the ultrasonic endoscope 12, and a monitor 20 that displays the ultrasound image and the endoscope image. The ultrasonography system 10 comprises a water supply tank 21a that stores cleaning water or the like, and a suction pump 21b that sucks aspirates inside the body cavity.

The ultrasonic endoscope 12 has an insertion part 22 that is inserted into the body cavity of the subject, an operating part 24 that is consecutively provided in a proximal end part of the insertion part 22 and is used by an operator to perform an operation, and a universal cord 26 that has one end connected to the operating part 24.

In the operating part 24, an air/water supply button 28a and a suction button 28b are provided side by side. In the operating part 24, a pair of angle knobs 29 and 29 and a treatment tool insertion port 30 are provided.

In the other end portion of the universal cord 26, an ultrasound connector 32a that is connected to the ultrasound processor device 14, an endoscope connector 32b that is connected to the endoscope processor device 16, and a light source connector 32c that is connected to the light source device 18 are provided. The ultrasonic endoscope 12 is attachably and detachably connected to the ultrasound processor device 14, the endoscope processor device 16, and the light source device 18 respectively through the connectors 32a, 32b, and 32c. The connector 32c comprises a water supply tube 34a that is connected to the water supply tank 21a, and a suction tube 34b that is connected to the suction pump 21b.

The insertion part 22 has, in order from a distal end side, a distal end part 40 that has an ultrasonic observation part 36 and an endoscope observation part 38, a bending part 42 that is connected to a proximal end side of the distal end part 40, and a soft part 44 that connects a proximal end side of the bending part 42 and a distal end side of the operating part 24. The distal end part 40, the bending part 42, and the soft part 44 are disposed along a longitudinal axis E of the insertion part 22. The bending part 42 is made by connecting a plurality of bending pieces (angle rings) and is configured to be freely bent. The soft part 44 is slender and long, and has flexibility.

The bending part 42 is remotely bent and operated by rotationally moving and operating a pair of angle knobs 29 and 29 provided in the operating part 24. With this, the distal end part 40 can be directed in a desired direction.

The ultrasound processor device 14 shown in FIG. 1 generates and supplies an ultrasound signal for making a plurality of ultrasound oscillators 48 of the ultrasound transducer 46 (see FIG. 2) configuring the ultrasonic observation part 36 generate ultrasonic waves. The ultrasound processor device 14 receives and acquires an echo signal reflected from an observation target part irradiated with the ultrasonic wave, by the ultrasound oscillator 48 and executes various kinds of signal processing on the acquired echo signal to generate an ultrasound image. The generated ultrasound image is displayed on the monitor 20. Here, the ultrasound transducer 46 corresponds to an ultrasound oscillator unit of the present invention.

The endoscope processor device 16 receives and acquires an image signal acquired from the observation target part illuminated with illumination light from the light source device 18 in the endoscope observation part 38 by the imaging element and executes various kinds of signal processing and image processing on the acquired image signal to generate an endoscope image. The generated endoscope image is displayed on the monitor 20.

In the example, the ultrasound processor device 14 and the endoscope processor device 16 are configured to two devices provided separately. Note that the present invention is not limited thereto, and both the ultrasound processor device 14 and the endoscope processor device 16 may be configured of one device.

The light source device 18 generates illumination light, such as white light or light of a specific wavelength. The illumination light propagates through a light guide (not shown) in the ultrasonic endoscope 12 and is emitted from the endoscope observation part 38 to illuminate an observation target part inside the body cavity.

The monitor 20 receives video signals generated by the ultrasound processor device 14 and the endoscope processor device 16 and displays an ultrasound image and an endoscope image. In regard to the display of the ultrasound image and the endoscope image, only one image may be appropriately switched and displayed on the monitor 20 or both images may be displayed simultaneously.

In the example, although the ultrasound image and the endoscope image are displayed on one monitor 20, a monitor for ultrasound image display and a monitor for endoscope image display may be provided separately. Alternatively, the ultrasound image and the endoscope image may be displayed in a display form other than the monitor 20, for example, in a form of being displayed on a display of a terminal carried with the operator.

Next, the configuration of the distal end part 40 will be described referring to FIG. 2. As shown in FIG. 2, the distal end part 40 is provided with the endoscope observation part 38 that acquires the endoscope image, on the distal end side, and the ultrasonic observation part 36 that acquires the ultrasound image, on the proximal end side.

The endoscope observation part 38 is provided with, on a distal end surface 50 of the distal end part 40, an observation window 52, illumination windows 54 and 54, an air/water supply nozzle 56, and a treatment tool outlet port 58. An observation optical system and an imaging element (not shown), such as a complementary metal oxide semiconductor (CMOS) or a charge coupled device (CCD), is disposed rearward of the observation window 52 (proximal end side) through a prism (not shown), and a signal cable is connected to a substrate that supports the imaging element. The signal cable is inserted into the insertion part 22, the operating part 24, the universal cord 26, and the like, extends to the connector 32b, and is connected to the endoscope processor device 16. Accordingly, an observation image fetched through the observation window 52 is formed on a light-receiving surface of the imaging element and is converted into an electric signal, and the electric signal is output to the endoscope processor device 16 through the signal cable and is converted into a video signal. With this, an endoscope image is displayed on the monitor 20 connected to the endoscope processor device 16.

An illumination optical system and an emission end of a light guide are disposed rearward of the illumination window 54 (proximal end side). The light guide is inserted into the insertion part 22, the operating part 24, and the universal cord 26, and an incidence end of the light guide is disposed in the connector 32c. Accordingly, the connector 32b is connected to the light source device 18, whereby illumination light emitted from the light source device 18 is transmitted to the illumination optical system through the light guide, and is emitted forward from the illumination window 54.

Air or cleaning water is ejected from the air/water supply nozzle 56 provided in the distal end part 40 toward the observation window 52. The above-described air or cleaning water is ejected from the air/water supply nozzle 56 by an operation on the air/water supply button 28a provided in the operating part 24.

The treatment tool outlet port 58 provided in the distal end part 40 is inserted into the treatment tool insertion port 30 through a treatment tool insertion channel (not shown). Accordingly, a treatment tool, such as a forcep, is inserted from the treatment tool insertion port 30, whereby it is possible to lead out the treatment tool from the treatment tool outlet port 58. The treatment tool outlet port 58 communicates with a suction pipe line (not shown) branched and connected to the treatment tool insertion channel, and the suction pipe line is connected to the suction pump 21b through the connector 32c and the suction tube 34b. The suction pipe line is opened and closed by the suction button 28b, whereby it is possible to suck a body fluid, such as blood, from the treatment tool outlet port 58.

As shown in FIG. 2, the ultrasonic observation part 36 is configured of the ultrasound transducer 46. The ultrasound transducer 46 is configured of a plurality of radial ultrasound oscillators 48 arranged in a circumferential direction on the outer peripheral surface of the distal end part 40. The ultrasound transducer 46 emits an ultrasonic wave toward a site to be observed and receives an echo signal of the ultrasonic wave. In performing an ultrasound tomographic surface examination, a balloon 60 is mounted on the outer peripheral surface of the distal end part 40 to cover the ultrasound transducer 46.

The outer peripheral surface of the distal end part 40 of the insertion part 22 of the ultrasonic endoscope 12 has balloon attachment grooves 90 for attachably and detachably attaching the balloon 60, formed along a peripheral direction of the outer peripheral surface. The balloon attachment grooves 90 have a first balloon attachment groove 92 provided on a proximal end side of the ultrasound transducer 46, and a second balloon attachment groove 94 provided on a distal end side of the ultrasound transducer 46.

The balloon 60 is made of an elastic material. As the elastic material, rubber made of silicone, rubber made of isoprene rubber, natural rubber, or the like can be used, and rubber made of silicone is preferably used. The balloon 60 has a balloon body 60A that has both ends provided with openings, a first annular locking part 60B and a second annular locking part 60C that are provided in the openings of the balloon body 60A and are locked in the first balloon attachment groove 92 and the second balloon attachment groove 94, respectively. The first annular locking part 60B and the second annular locking part 60C are formed in an annular shape narrowed with respect to the balloon body 60A.

The balloon 60 is inserted into the insertion part 22, whereby the first annular locking part 60B is disposed in the first balloon attachment groove 92 and the second annular locking part 60C is disposed in the second balloon attachment groove 94. The first balloon attachment groove 92 and the second balloon attachment groove 94 are provided to match the first annular locking part 60B and the second annular locking part 60C of the balloon 60 to be mounted, and the first annular locking part 60B and the second annular locking part 60C are fitted into the first balloon attachment groove 92 and the second balloon attachment groove 94, respectively. The first annular locking part 60B and the second annular locking part 60C are configured to have an inner diameter smaller than an outer diameter of the distal end part 40 of the ultrasonic endoscope 12 in a state before mounting. In a case where the balloon 60 is mounted on the distal end part 40 or after the balloon 60 is mounted, the first annular locking part 60B and the second annular locking part 60C are expanded in diameter compared to the above-described state before mounting, elastic force of the first annular locking part 60B and elastic force of the second annular locking part 60C are applied toward an inside of the distal end part 40 in a radial direction. That is, the balloon 60 is mounted on the distal end part 40, whereby the widened first annular locking part 60B and second annular locking part 60C contract to return to an original size. With contractile force of the first annular locking part 60B and the second annular locking part 60C, the balloon 60 is maintained at a predetermined position of the distal end part 40.

The distal end part 40 has a supply/discharge port 96 has an outlet/inlet of a communication path (not shown) on the proximal end side of the ultrasound transducer 46 and on a distal end side of the first balloon attachment groove 92. The supply/discharge port 96 is opened in an internal space of the balloon 60 attached to the balloon attachment grooves 90. A fluid is supplied to or discharged from the internal space of the balloon 60 through the communication path and the supply/discharge port 96. Examples of the fluid include deaerated water as an ultrasonic wave transmission medium. The balloon is made to expand by supplying the fluid into the balloon 60, and is brought into contact with a site to be observed inside a body. With this, air is eliminated from a region between the site to be observed and the ultrasound transducer 46, that is, a scanning region of an ultrasonic wave, and attenuation of the ultrasonic wave and the echo signal is restrained. In extracting the insertion part 22 from the body of the subject, the fluid in the balloon 60 is discharged from the supply/discharge port 96 to contract the balloon 60.

The supply and the suction of the fluid to the balloon 60 are performed by the air/water supply button 28a and the suction button 28b provided in the operating part 24, respectively. A configuration for performing the supply and the suction of the fluid of the balloon 60 using the air/water supply button 28a and the suction button 28b is known in JP2020-171435A and the like. Thus, detailed description thereof will not be repeated, and an outline will be given as described below.

The air/water supply button 28a is a two-stage switchable button. An air outlet (not shown) that communicates with the atmosphere is formed at an operation cap of the air/water supply button 28a, and in a case where the air/water supply button 28a is not operated, air from an air supply pump (not shown) leaks from the air outlet. Then, in a case where the air outlet is closed in this state, air from the air supply pump is ejected from the air/water supply nozzle 56.

On the other hand, in a case where a one-stage push operation of the air/water supply button 28a is performed, water from the water supply tank 21a is ejected from the air/water supply nozzle 56. Then, in a case where a two-stage push operation of the air/water supply button 28a is performed, water from the water supply tank 21a passes through the communication path and is supplied from the supply/discharge port 96 into the balloon 60. With this, the balloon 60 expands by water. It is preferable that deaerated water is stored in the water supply tank 21a to be usable as an ultrasonic wave transmission medium.

The suction button 28b is also a two-stage switchable button like the air/water supply button 28a. In a case where a one-stage push operation of the suction button 28b is performed, the treatment tool insertion channel is connected to the suction pump, and the body fluid, such as blood, is sucked from the treatment tool outlet port 58. Then, in a case where a two-stage push operation of the suction button 28b is performed, the communication path is connected to the suction pump 21b, and water in the balloon 60 is sucked and discharged from the supply/discharge port 96. With this, the balloon 60 contracts.

### Configuration of Attachment Part of Distal End Part and Balloon

Next, the configuration of an attachment part of the distal end part 40 of the ultrasonic endoscope 12 and the balloon 60 will be described. In a case of being mounted on the distal end part 40 of the ultrasonic endoscope 12 and used, the balloon 60 is required to be resistant to (1) leakage of a fluid from the end portion of the balloon 60 (fluid leakage resistance) and (2) detachment from the balloon 60 during use (balloon detachment resistance). To improve the fluid leakage resistance, there is a need to firmly fix the distal end part 40 with the first annular locking part 60B and the second annular locking part 60C. Examples of a method of firmly fixing the distal end part 40 include a method of increasing the volume of the first annular locking part 60B and the second annular locking part 60C or a method of improving the contractile force of the first annular locking part 60B and the second annular locking part 60C. Note that, in a case where the volume of the first annular locking part 60B and the second annular locking part 60C increases, portions of the first annular locking part 60B and the second annular locking part 60C protruding from the first balloon attachment groove 92 and the second balloon attachment groove 94 are made large. For this reason, in inserting or extracting the insertion part 22 into or from the subject, the first annular locking part 60B and the second annular locking part 60C may be brought into contact with a wall portion in the subject, the balloon 60 may be likely to be detached, and the balloon detachment resistance may be degraded.

In the present invention, a detachment prevention shape described below is provided in the first annular locking part 60B and the second annular locking part 60C of the balloon 60 that are locked in the first balloon attachment groove 92 and the second balloon attachment groove 94, thereby achieving both improvement of the fluid leakage resistance and the improvement of the balloon detachment resistance. Hereinafter, each embodiment will be described.

### First Embodiment

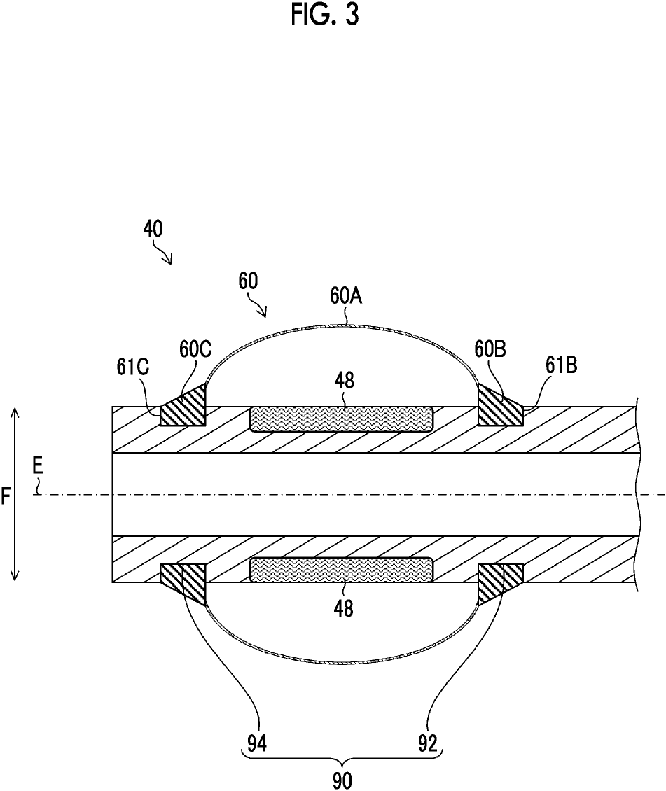

FIG. 3 is a schematic view (side sectional view) of a distal end part 40 of an ultrasonic endoscope 12 of a first embodiment. In a case where the balloon 60 is mounted on the distal end part 40, an axial direction (a direction perpendicular to a peripheral direction of the first annular locking part 60B and the second annular locking part 60C) of the balloon 60 coincides with a longitudinal axis (a longitudinal axis of the distal end part 40) E of the insertion part 22. In the following description, it is assumed that the "balloon axial direction" means the same direction as the longitudinal axis E of the distal end part.

The distal end part 40 of the ultrasonic endoscope 12 of the first embodiment has the balloon 60 in which a detachment prevention shape is provided in the first annular locking part 60B and the second annular locking part 60C. The first annular locking part 60B and the second annular locking part 60C have, as the detachment prevention shape, a shape in which a thickness on the balloon body 60A side is the thickest and the thickness is thinned toward end parts 61B and 61C on an opposite side to the balloon body 60A, in a cross-sectional shape in a case where the first annular locking part 60B and the second annular locking part 60C are cut in the balloon axial direction. Hereinafter, although the first annular locking part 60B and the first balloon attachment groove 92 will be described, the second annular locking part 60C and the second balloon attachment groove 94 also have the same configuration.

As shown in FIG. 3, the first balloon attachment groove 92 is formed to have a rectangular shape in the cross section taken in the balloon axial direction (a direction of the longitudinal axis E). Then, an inside of the first annular locking part 60B in a radial direction F perpendicular to the balloon axial direction is fitted into the groove shape of the first balloon attachment groove 92. With this, the first annular locking part 60B is formed to be locked in the first balloon attachment groove 92, such that it is possible to restrain fluid leakage.

On the other hand, an outside of the first annular locking part 60B in the radial direction F has an inclined structure to be gradually inclined to the inside in the radial direction F from the balloon body 60A side toward the end portion 61B on the opposite side to the balloon body 60A. The outside of the first annular locking part 60B has the inclined structure from the balloon body 60A side toward the end portion 61B side on the opposite side, whereby the thickness of the first annular locking part 60B is thickened on the balloon body 60A side and is thinned in the end portion 61B on the opposite side.

As the first annular locking part 60B is formed in such a shape, in inserting or extracting the insertion part 22 of the ultrasonic endoscope 12 into or from the subject, in a case where force of a component in the balloon axial direction is applied to the first annular locking part 60B, it is possible to disperse the force to the outside of the first annular locking part 60B in the radial direction F along the above-described inclined structure. Since the second annular locking part 60C also has the same configuration (inclined structure) as the first annular locking part 60B, in inserting or extracting the insertion part 22 of the ultrasonic endoscope 12 into or from the subject, it is possible to disperse force applied to the second annular locking part 60C. Accordingly, since it is possible to reduce force applied to the first annular locking part 60B and the second annular locking part 60C in the balloon axial direction, it is possible to restrain the first annular locking part 60B and the second annular locking part 60C from being detached from the first balloon attachment groove 92 and the second balloon attachment groove 94, respectively. Therefore, it is possible to restrain the balloon 60 from being detached from the distal end part 40.

The cross section of the first balloon attachment groove 92 taken in the balloon axial direction is not limited to the rectangular shape, and may be a trapezoid or a rhombus or may be a polygon, such as a pentagon. In this case, a portion of the first annular locking part 60B fitting into the first balloon attachment groove 92 has a shape corresponding to the first balloon attachment groove 92. With this, it is possible to easily lock the first annular locking part 60B in the first balloon attachment groove 92. The same applies to the second balloon attachment groove 94.

In this way, with the ultrasonic endoscope 12 of the first embodiment, in inserting or extracting the insertion part 22 into or from the subject, even though the first annular locking part 60B and the second annular locking part 60C are brought into contact with a wall surface in the subject, it is possible to disperse force applied to the first annular locking part 60B and the second annular locking part 60C. With this, it is possible to restrain the first annular locking part 60B and the second annular locking part 60C from being detached from the first balloon attachment groove 92 and the second balloon attachment groove 94, and to restrain the balloon 60 from being detached from the distal end part 40. The thickness of each of the first annular locking part 60B and the second annular locking part 60C on the balloon body 60A side is thickened, whereby it is possible to improve contractile force and to improve the fluid leakage resistance.

### Second Embodiment

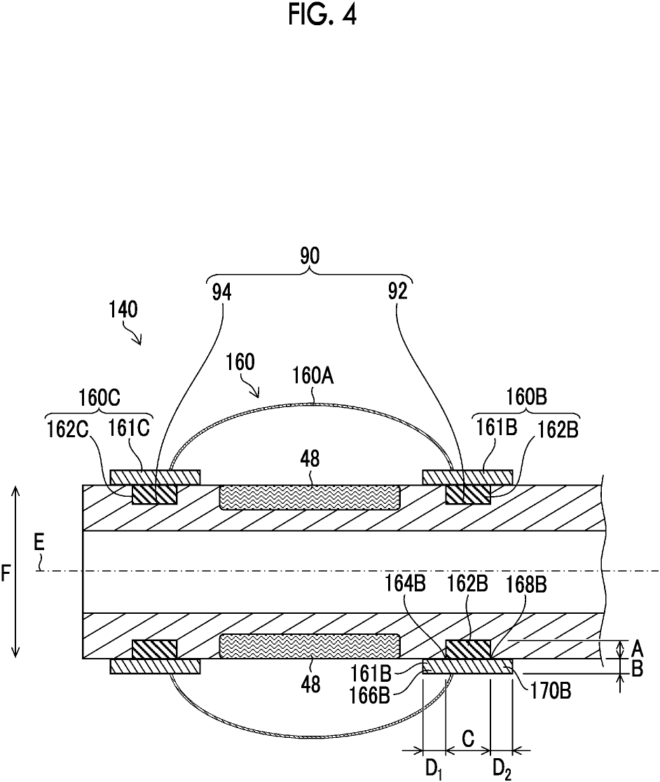

FIG. 4 is a schematic view (side sectional view) of a distal end part 140 of an ultrasonic endoscope 12 of a second embodiment. In the distal end part 140 of the ultrasonic endoscope 12 of the second embodiment, a first annular locking part 160B of a balloon 160 has a toric part 161B that is formed in a toric shape in plan view from the balloon axial direction, and a toric protrusion part 162B that is formed along the toric part 161B and protrudes in an inward direction from the toric part 161B. A second annular locking part 160C has a toric part 161C that is formed in a toric shape in plan view from the balloon axial direction, and a toric protrusion part 162C that is formed along the toric part 161C and protrudes in an inward direction from the toric part 161C. The toric part 161B and the toric part 161C are connected to a balloon body 160A and are positioned on an outside surface of the distal end part 140. The toric protrusion parts 162B and 162C have shapes that are fitted into the first balloon attachment groove 92 and the second balloon attachment groove 94, respectively. In the ultrasonic endoscope 12 of the second embodiment, a detachment prevention shape includes the toric parts 161B and 161C and the toric protrusion parts 162B and 162C. Hereinafter, although the first annular locking part 160B will be described, the second annular locking part 160C also has the same configuration.

As shown in FIG. 4, the first annular locking part 160B is configured such that, in a cross-sectional shape taken in the balloon axial direction, the toric part 161B is formed in a rectangular shape, is brought into contact with the outside surface of the distal end part 140, and fixes the outside surface of the distal end part 140 by contractile force. The toric protrusion part 162B is disposed on the inside of the toric part 161B in a radial direction F and is provided in a step shape with respect to the toric part 161B.

As shown in FIG. 4, in the cross-sectional shape in a case where the first annular locking part 160B is cut in the balloon axial direction, in a case where the balloon axial direction is set as a width direction, the toric part 161B is formed to be wider than the toric protrusion part 162B. The toric part 161B has an internal extension part 166B that extends from a protruding position 164B on the balloon body 160A side that is a position where the toric protrusion part 162B protrudes from the toric part 161B, toward the balloon body 160A side on an outer peripheral surface of the distal end part 140. The toric part 161B has an external extension part 170B that extends from a protruding position 168B on an opposite side to the balloon body 160A that is a position where the toric protrusion part 162B protrudes from the toric part 161B, toward the opposite side to the balloon body 160A on the outer peripheral surface of the distal end part 140.

In this way, the toric part 161B is formed to be wider than the toric protrusion part 162B, whereby the first annular locking part 160B does not fasten the distal end part 140 only at the position of the first balloon attachment groove 92, and the distal end part 140 can be fastened by contraction in a wide range of the outside surface of the distal end part 140. Accordingly, it is possible to firmly fix the balloon 160 to the distal end part 140, and to restrain fluid leakage.

In a case where the balloon axial direction is set as a width direction, it is preferable that, in a case where a width of the toric protrusion part 162B is C, a width of the internal extension part 166B is D₁, and a width of the external extension part 170B is D₂, a width of each of the internal extension part 166B and the external extension part 170B of the toric part 161B satisfied the following expressions.

$$\text{C/2} \leq {\text{D}}_{1} \leq 2 \text{C}$$

$$\text{C/2} \leq {\text{D}}_{2} \leq 2 \text{C}$$

As the above-described expressions are satisfied, it is possible to secure fastening force for fastening the distal end part 140 with the internal extension part 166B and the external extension part 170B of the toric part 161B. End parts of the internal extension part 166B and the external extension part 170B are turned up, whereby it is possible to restrain the detachment of the toric part 161B.

As one preferred aspect, in FIG. 4, although a shape in which the toric part 161B of the first annular locking part 160B has both the internal extension part 166B and the external extension part 170B has been shown, the toric part 161B does not always have both the internal extension part 166B and the external extension part 170B, and may be formed in a shape having any one of the internal extension part 166B or the external extension part 170B. As the toric part 161B has the internal extension part 166B, it is possible to fix the balloon body 160A side, and to restrain fluid leakage in the balloon 160. As the toric part 161B has the external extension part 170B, it is possible to easily attach the balloon 160 by holding the external extension part 170B and attaching the balloon 160 to the distal end part.

In a case where the radial direction F perpendicular to the balloon axial direction is set as a height direction, it is preferable that a relationship of a height of the toric protrusion part 162B greater than a height of the toric part 161B is satisfied. That is, in a case where the height of the toric protrusion part 162B is A, and the height of the toric part 161B is B, it is preferable that the following expression is satisfied.

$$\text{B<A}$$

As the above-described expression is satisfied, in inserting or extracting the insertion part 22 into or from the subject, even in a case where a wall portion of the subject is brought into contact with the toric part 161B, the toric protrusion part 162B is hardly pulled out from the first balloon attachment groove 92. Since the height of the toric part 161B can be reduced, the wall portion of the subject is hardly brought into contact with the toric part 161B. With this, since it is possible to restrain force from being applied to the first annular locking part 160B, it is possible to restrain the first annular locking part 160B from detached from the first balloon attachment groove 92.

In attaching the first annular locking part 160B to the first balloon attachment groove 92, in a case where the toric part 161B is in contact with the outside surface of the distal end part 140, the height of the toric protrusion part 162B is not limited. As shown in FIG. 4, it is preferable that the height of the toric protrusion part 162B is equal to a depth of the first balloon attachment groove 92. As the height of the toric protrusion part 162B is made to be equal to the depth of the first balloon attachment groove 92, since it is possible to bring the toric part 161B into contact with the outside surface of the distal end part 140 of the ultrasonic endoscope 12 in a wide range, it is possible to fix the balloon 160 to the distal end part 140 by the contractile force of the first annular locking part 160B.

In this way, in the ultrasonic endoscope 12 of the second embodiment, the first annular locking part 160B has a configuration in which the toric part 161B is wider than the width of the first balloon attachment groove 92 in the width direction. The second annular locking part 160C also has the same configuration as the first annular locking part 160B. Accordingly, contact areas of the first annular locking part 160B and the second annular locking part 160C with the distal end part 140 increase, whereby it is possible to firmly fix the balloon 160 to the distal end part 140, and even though the heights of the toric parts 161B and 161C are reduced, it is possible to fix the balloon 160. As the heights of the toric parts 161B and 161C are reduced, since it is possible to suppress application of force of a component in the balloon axial direction to the first annular locking part 160B and the second annular locking part 160C, it is possible to restrain the first annular locking part 160B and the second annular locking part 160C from being detached from the first balloon attachment groove 92 and the second balloon attachment groove 94, respectively. With this, it is possible to restrain the balloon 160 from being detached from the distal end part 140.

### Third Embodiment

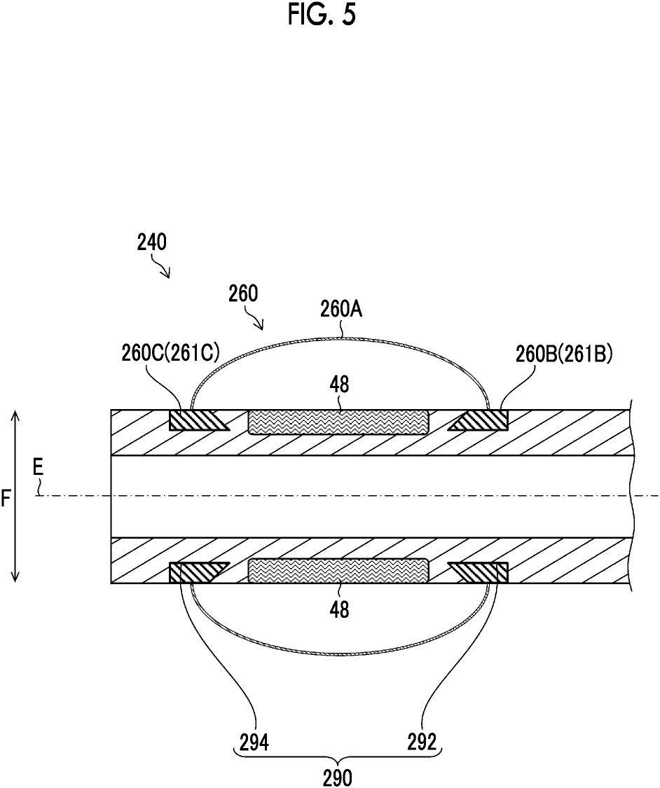

FIG. 5 is a schematic view (side sectional view) of a distal end part 240 of an ultrasonic endoscope 12 of a third embodiment. The distal end part 240 of the ultrasonic endoscope 12 of the third embodiment has a balloon 260 in which a detachment prevention shape is provided in a first annular locking part 260B and a second annular locking part 260C. In a cross-sectional shape in a case where the first annular locking part 260B and the second annular locking part 260C are cut in the balloon axial direction, in a case where the balloon axial direction is set as a width direction, the first annular locking part 260B and the second annular locking part 260C have, as the detachment prevention shape, fitting shape part 261B and 261C that an inside in the radial direction F is formed to be wider than an outside. A first balloon attachment groove 292 and a second balloon attachment groove 294 of the distal end part 240 have groove shapes into which the fitting shape parts 261B and 261C are fitted in the width direction with no gap. Hereinafter, although the first annular locking part 260B will be described, the second annular locking part 260C also has the same configuration.

As shown in FIG. 5, the first annular locking part 260B of the balloon 260 has the fitting shape part 261B in which the inside in the radial direction F is wider than the outside in the width direction. Then, the first balloon attachment groove 292 is formed in a reversed shape of the fitting shape part 261B. That is, the first balloon attachment groove 292 is configured such that a bottom portion of the first balloon attachment groove 292 in a depth direction is formed to have a wide width, and an opening portion of the first balloon attachment groove 292 is formed to have a narrow width. With this, the first annular locking part 260B is hardly detached from the first balloon attachment groove 292, and it is possible to restrain the balloon 260 from being detached from the distal end part 240.

The fitting shape part 261B has a shape in which the inside in the radial direction F having a wide width is widened to the balloon body 260A side from the outside toward the inside in the radial direction F. That is, the first annular locking part 260B (fitting shape part 261B) has an inclined structure to be inclined to the balloon body 260A side from the outside toward the inside in the radial direction F. The first balloon attachment groove 292 has an inclined structure in which the balloon body 260A side (ultrasound oscillator 48 side) is widened to the ultrasound oscillator 48 side from the opening portion toward the bottom portion of the first balloon attachment groove 292, to correspond to the inclined structure of the fitting shape part 261B. In a case where the inclined structure of the fitting shape part 261B is brought into contact with the inclined structure of the first balloon attachment groove 292, and the force of the component in the balloon axial direction (force toward the balloon body 60A side) is applied to the first annular locking part 260B, the force is dispersed to the bottom portion side of the first balloon attachment groove 292. Thus, it is possible to restrain the detachment of the first annular locking part 260B, and to restrain the balloon 260 from being pulled out.

In a case where the radial direction F of the first annular locking part 260B is set as a height direction, it is preferable that a height of the first annular locking part 260B is equal to or less than a depth of the first balloon attachment groove 292. That is, it is preferable that the first annular locking part 260B is within the first balloon attachment groove 292 and does not protrude from the first balloon attachment groove 292. In particular, it is preferable that the height of the first annular locking part 260B coincides with the depth of the first balloon attachment groove 292. As the protrusion of the first annular locking part 260B from the first balloon attachment groove 292 is restrained, even though the force of the component in the balloon axial direction is applied, it is possible to restrain contact with the first annular locking part 260B, and to restrain the detachment of the first annular locking part 260B. As the height of the first annular locking part 260B coincides with the depth of the first balloon attachment groove 292, it is possible to restrain the force of the component in the balloon axial direction from being applied to the first annular locking part 260B, and to fix the balloon 260 to the distal end part 240 by the contractile force of the first annular locking part 260B.

In this way, the ultrasonic endoscope 12 of the third embodiment has a configuration in which the fitting shape part 261B with the inside of the first annular locking part 260B in the radial direction F having a wide width is provided, and the shape of the first balloon attachment groove 292 is made to correspond to the fitting shape part 261B. The second annular locking part 260C and the second balloon attachment groove 294 also have the same configurations as the first annular locking part 260B and the first balloon attachment groove 292. Accordingly, it is possible to suppress the detachment of the first annular locking part 260B and the second annular locking part 260C from the first balloon attachment groove 292 and the second balloon attachment groove 294, respectively, and to restrain the balloon 260 from being detached from the distal end part 240. Since the first balloon attachment groove 292 and the fitting shape part 261B, and the second balloon attachment groove 294 and the fitting shape part 261C are fitted in the width direction with no gap, it is possible to improve the fluid leakage resistance of the fluid in the balloon 260.

### Fourth Embodiment

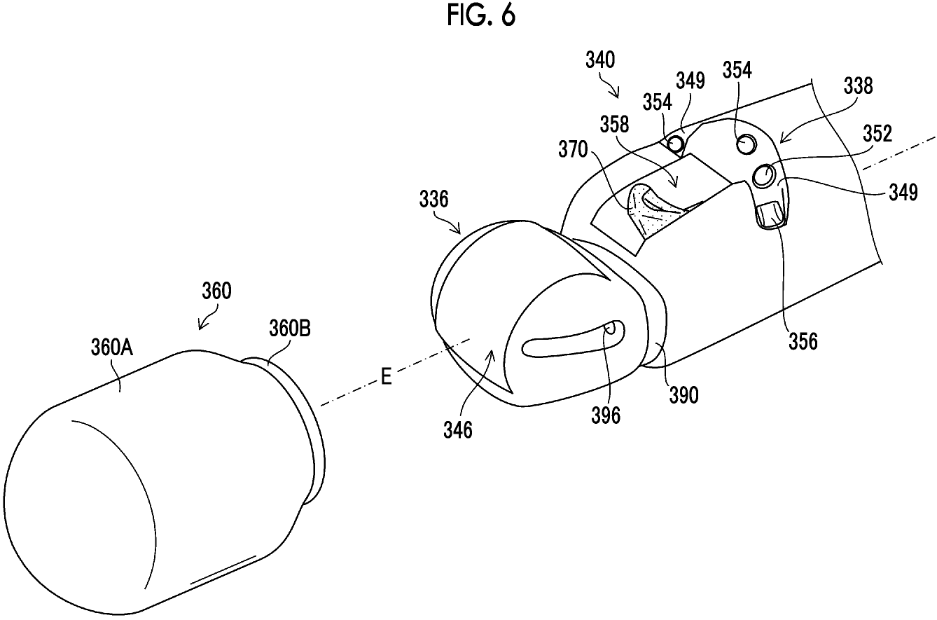

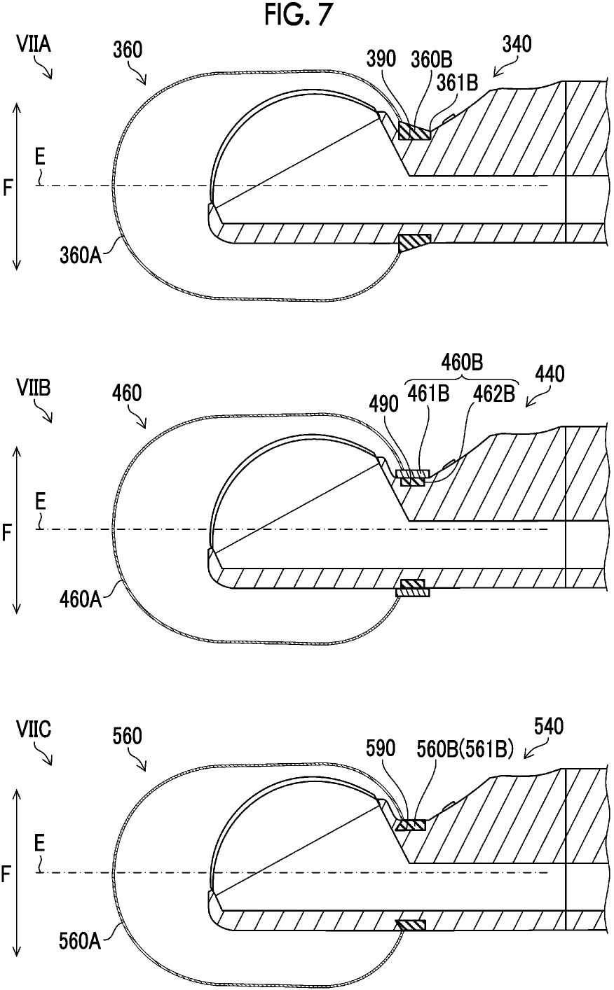

FIGS. 6 and 7 are diagrams illustrating an ultrasonic endoscope 12 of a fourth embodiment. FIG. 6 is a partial enlarged perspective view showing the appearance of an example of a distal end part 340 of the ultrasonic endoscope 12 of the fourth embodiment. FIG. 7 is a schematic view (side sectional view) of distal end parts 340, 440, and 540 of the ultrasonic endoscope of the fourth embodiment.

As shown in FIG. 6, the distal end part 340 of the ultrasonic endoscope 12 is provided with an ultrasonic observation part 336 that acquires an ultrasound image, on a distal end side, and an endoscope observation part 338 that acquires an endoscope image, on a proximal end side. A balloon attachment groove 390 to which a balloon 360 is attachably and detachably attached is provided between the ultrasonic observation part 336 and the endoscope observation part 338 of the distal end part 340.

The ultrasonic observation part 336 has a convex type ultrasound transducer 346 in which a large number of ultrasound oscillators that transmit and receive ultrasonic waves are arranged in a convex shape. The ultrasonic observation part 336 is provided with a supply/discharge port 396 that supplies and discharges a fluid to and from the balloon 360.

The endoscope observation part 338 has an observation window 352 and illumination windows 354 and 354, and the observation window 352 and the illumination windows 354 and 354 are disposed on an inclined surface portion 349 that is formed to be inclined with respect to a direction of a longitudinal axis E of the insertion part 22 from the balloon attachment groove 390 toward a proximal end side in the distal end part 340. The inclined surface portion 349 is provided with an air/water supply nozzle 356 near the observation window 352. The air/water supply nozzle 356 ejects water or air toward the observation window 352 to clean the observation window 352.

The distal end part 340 is provided with a treatment tool outlet port 358. One end of a treatment tool insertion channel inserted into the insertion part 22 is connected to the treatment tool outlet port 358, and a treatment tool inserted into a treatment tool insertion port 30 is introduced from the treatment tool outlet port 358 into the subject through the treatment tool insertion channel.

Inside the treatment tool outlet port 358, an elevator 370 that varies a lead-out direction of the treatment tool introduced from the treatment tool outlet port 358 into the subject is provided. One end of a wire is connected to the elevator 370 through an elevating drive member, and the other end of the wire is connected to an elevating lever (not shown) of the operating part 24. An elevation angle of the elevator 370 changes with a push/pull operation of the wire by an operation on the elevating lever, and accordingly, the treatment tool is led out in a desired direction.

The balloon 360 that is attached to the distal end part 340 of the ultrasonic endoscope 12 of the fourth embodiment has a balloon body 360A that has one end provided with an opening and the other end closed, and an annular locking part 360B that is provided in the opening of the balloon body 360A. The balloon attachment groove 390 provided in the distal end part 340 is configured of an annular groove portion that is formed along a peripheral direction of an outer peripheral surface of the distal end part 340. The annular locking part 360B of the balloon 360 is locked in the balloon attachment groove 390, whereby the balloon 360 is attached to the distal end part 340. The annular locking part 360B has a detachment prevention shape, whereby the annular locking part 360B is restrained from being detached from the balloon attachment groove 390.

### Configuration of Detachment Prevention Shape

Next, detachment prevention shapes provided in annular locking parts 360B, 460B, and 560B of balloons 360, 460, and 560 will be described referring to FIG. 7.

Reference numeral VIIA of FIG. 7 indicates the distal end part 340 of the ultrasonic endoscope 12 in which the detachment prevention shape of the annular locking part 360B has the same configuration as in the above-described first embodiment. That is, in a cross-sectional shape of the annular locking part 360B taken in the balloon axial direction, the annular locking part 360B has, as the detachment prevention shape, a shape in which a thickness of the annular locking part 360B on the balloon body 360A side is the thickest, and the thickness is thinned toward an end portion 361B on an opposite side to the balloon body 360A. With this, in extracting the ultrasonic endoscope 12 from the subject, in a case where force of a component in the balloon axial direction is applied to the annular locking part 360B, it is possible to disperse the force in an outward direction of the radial direction F of the annular locking part 360B. Thus, it is possible to restrain the annular locking part 360B from being detached from the balloon attachment groove 390.

Since a shape of the inside of the annular locking part 360B in the radial direction F is a shape that is fitted into a groove shape of the balloon attachment groove 390, as the annular locking part 360B is locked in the balloon attachment groove 390, the balloon 360 is fixed to the distal end part 340.

Reference numeral VIIB of FIG. 7 indicates the distal end part 440 of the ultrasonic endoscope 12 in which the detachment prevention shape of the annular locking part 460B has the same configuration as in the above-described second embodiment. That is, the annular locking part 460B has, as the detachment prevention shape, a toric part 461B that is positioned on the outside surface of the distal end part 440, and a toric protrusion part 462B that protrudes in an inward direction of the toric part 461B. As the toric part 461B is provided on the outside surface of the distal end part 440, since it is possible to fix the toric part 461B to the distal end part 440 by the contractile force of the toric part 461B in a wide range, it is possible to firmly fix the balloon 460.

As the toric protrusion part 462B has a shape that is fitted into the balloon attachment groove 490, and the toric protrusion part 462B is locked in the balloon attachment groove 490, the balloon 460 is fixed to the distal end part 440.

Reference numeral VIIC of FIG. 7 indicates the distal end part 540 of the ultrasonic endoscope 12 in which the detachment prevention shape of the annular locking part 560B has the same configuration as in the above-described third embodiment. That is, in a cross-sectional shape of the annular locking part 560B taken in the balloon axial direction, the annular locking part 560B has, as the detachment prevention shape, a fitting shape part 561B in which an inside in the radial direction F is formed to be wider than an outside. In a case where the balloon axial direction is set as a width direction, a balloon attachment groove 590 of the distal end part 540 of the ultrasonic endoscope 12 has a groove shape into which the fitting shape part 561B of the annular locking part 560B is fitted in the width direction with no gap. With this, since the fitting shape part 561B has a shape that is widened from the outside toward the inside in the radial direction F of the annular locking part 560B, and the balloon attachment groove 590 has a shape that has a narrow opening portion and is widened toward the bottom portion, it is possible to restrain the annular locking part 560B from being detached from the balloon attachment groove 90.

In this way, as in the fourth embodiment, in regard to the ultrasonic endoscope 12 having the convex type ultrasound transducer 346, the detachment prevention shape for restraining the annular locking part 360B, 460B, or 560B from being detached from the balloon attachment groove 390, 490, or 590 of the distal end part 340, 440, or 540 is provided in the balloon 360, 460, or 560, whereby it is possible to restrain the detachment of the balloon 360, 460, or 560 from the distal end part 340, 440, or 540, and to improve the balloon detachment resistance.

### Manufacturing Method of Balloon

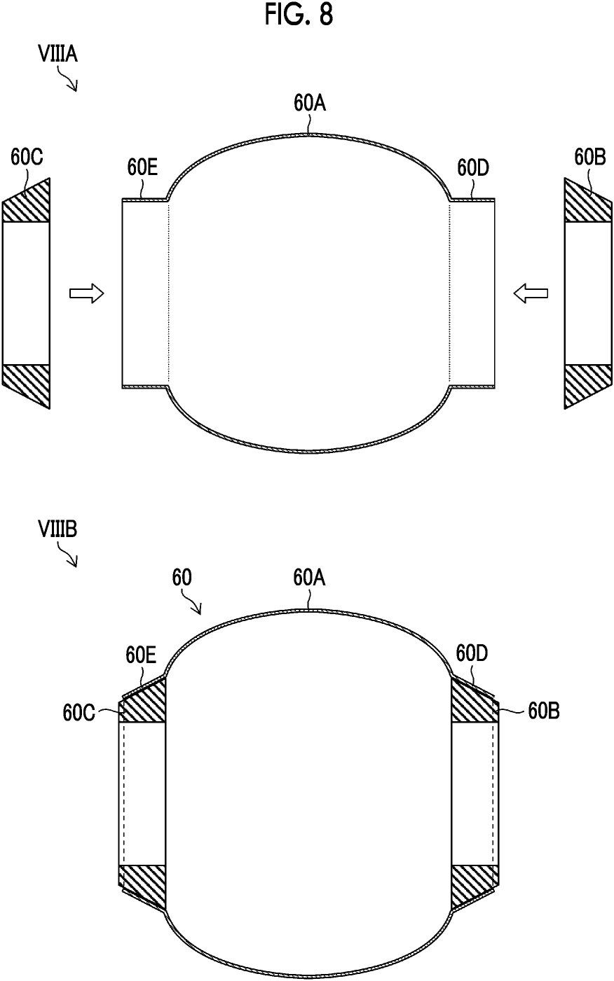

Next, a manufacturing method of a balloon 60 will be described with the balloon 60 of the above-described first embodiment as an example. FIG. 8 is a diagram illustrating a manufacturing method of the balloon 60.

As indicated by reference numeral VIIIA of FIG. 8, the balloon 60 has the balloon body 60A, the first annular locking part 60B, and the second annular locking part 60C that are formed separately.

A manufacturing method of each of the balloon body 60A, the first annular locking part 60B, and the second annular locking part 60C is not particularly limited. As the manufacturing method of the balloon body 60A, for example, a mold is immersed into a solution of resin or rubber that is a material of the balloon body 60A, is then taken out from the solution, and is dried. The balloon body 60A is formed in the periphery of the mold accordingly. Thereafter, the mold is taken out from the inside of the balloon body 60A, whereby the balloon body 60A can be manufactured. The first annular locking part 60B or the second annular locking part 60C can be manufactured by forming an inside of a mold in a shape of the first annular locking part 60B or the second annular locking part 60C, filling the inside of the mold with a solution of resin or rubber that is a material of the first annular locking part 60B or the second annular locking part 60C, solidifying the solution, and taking out a solidified product from the mold.

After the balloon body 60A, the first annular locking part 60B, and the second annular locking part 60C are formed, as indicated by reference numeral VIIIB, the members are bonded to manufacture the balloon 60. The members can be bonded by adhesion using an adhesive. The members can be bonded by welding for melting the members by heating a bonding position of the balloon body 60A and the first annular locking part 60B and a bonding position of the balloon body 60A and the second annular locking part 60C, then solidifying the member, and bonding the members.

The bonding position of the balloon body 60A and the first annular locking part 60B and the bonding position of the balloon body 60A and the second annular locking part 60C are bonded such that the first annular locking part 60B and the second annular locking part 60C are disposed on an inner surface side of the balloon body 60A. That is, bead parts 60D and 60E of the balloon body 60A are bonded to the outside of the members of the first annular locking part 60B and the second annular locking part 60C. As the first annular locking part 60B and the second annular locking part 60C are disposed on the inner surface side of the balloon body 60A, since it is possible to bond the balloon body to the first annular locking part 60B and the second annular locking part 60C in a state in which the bead parts 60D and 60E of the balloon 60 are extended, it is possible to facilitate manufacturing.

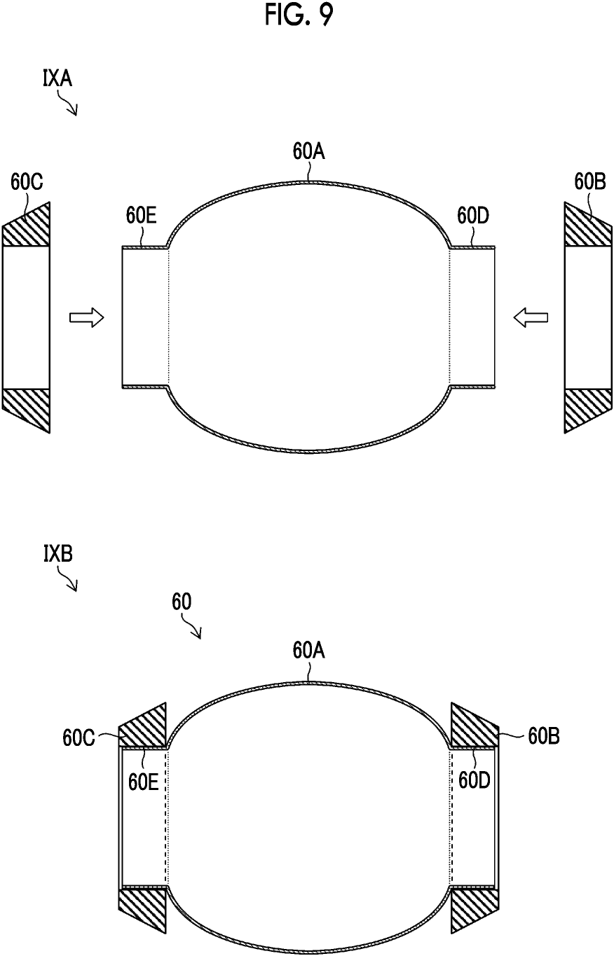

The bonding position of the balloon body 60A and the first annular locking part 60B and the bonding position of the balloon body 60A and the second annular locking part 60C are not limited to the inner surface side of the balloon body 60A shown in FIG. 8. FIG. 9 is a diagram illustrating another manufacturing method of a balloon 60. Even in the manufacturing method of the balloon 60 shown in FIG. 9, as indicated by reference numeral IXA, the balloon 60 has the balloon body 60A, the first annular locking part 60B, and the second annular locking part 60C are formed separately.

Thereafter, as indicated by reference numeral IXB, the inside of the first annular locking part 60B and the second annular locking part 60C are bonded to an outer surface side of the bead parts 60D and 60E of the balloon body 60A. As the first annular locking part 60B and the second annular locking part 60C are bonded to the outside of the balloon body 60A, in a case where the balloon 60 is mounted on the distal end part 40, it is possible to dispose the bead parts 60D and 60E of the balloon body 60A between the first annular locking part 60B and the first balloon attachment groove 92 and between the second annular locking part 60C and the second balloon attachment groove 94. Accordingly, since it is possible to bring the balloon body 60A into contact with the distal end part 40, it is possible to improve the fluid leakage resistance of the fluid from the balloon 60.

In a case where silicone is used as the material of the balloon body 60A, it is difficult to form an annular locking part by rounding the bead parts 60D and 60E of the balloon body 60A. Accordingly, the balloon body 60A is formed separately from the first annular locking part 60B and the second annular locking part 60C, and the balloon body 60A, the first annular locking part 60B and the second annular locking part 60C are integrated, whereby it is possible to manufacture a balloon using rubber made of silicone.

In FIGS. 8 and 9, although the method of manufacturing the balloon 60 of the first embodiment described above has been described, the balloon 160 of the second embodiment and the balloon 360 or 460 of the fourth embodiment described above can be manufactured by the same method as the manufacturing method shown in FIG. 8 in which the balloon body is bonded to the annular locking parts such that the annular locking parts are disposed on the inner surface side of the balloon body. The balloon 260 of the third embodiment and the balloon 360 or 560 of the fourth embodiment described above can be manufactured by the same method as the manufacturing method shown in FIG. 9 in which the balloon body is bonded to the annular locking parts such that the annular locking parts are disposed on the outer surface side of the balloon body. In regard to the balloon 160 of the second embodiment, in a case where the toric part 161B of the first annular locking part 160B has only the external extension part 170B and does not have the internal extension part 166B, the balloon 160 can be manufactured by the manufacturing method shown in FIG. 9. The same applies to the balloon 460 of the fourth embodiment.

### EXPLANATION OF REFERENCES

10: ultrasonography system
12: ultrasonic endoscope
14: ultrasound processor device
16: endoscope processor device
18: light source device
20: monitor
21 a: water supply tank
21b: suction pump
22: insertion part
24: operating part
26: universal cord
28a: air/water supply button
28b: suction button
29: angle knob
30: treatment tool insertion port
32a, 32b, 32c: connector
34a: water supply tube
34b: suction tube
36: ultrasonic observation part
38: endoscope observation part
40: distal end part
42: bending part
44: soft part
46: ultrasound transducer
48: ultrasound oscillator
50: distal end surface
52: observation window
54: illumination window
56: air/water supply nozzle
58: treatment tool outlet port
60: balloon
60A: balloon body
60B: first annular locking part
60C: second annular locking part
60D, 60E: bead part
61B, 61C: end part
90: balloon attachment groove
92: first balloon attachment groove
94: second balloon attachment groove
96: supply/discharge port
140: distal end part
160: balloon
160A: balloon body
160B: first annular locking part
160C: second annular locking part
161B: toric part
161C: toric part
162B: toric protrusion part
162C: toric protrusion part
164B: protruding position
166B: internal extension part
168B: protruding position
170B: external extension part
240: distal end part
260: balloon
260A: balloon body
260B: first annular locking part
260C: second annular locking part
261B: fitting shape part
261C: fitting shape part
292: first balloon attachment groove
294: second balloon attachment groove
336: ultrasonic observation part
338: endoscope observation part
340: distal end part
346: ultrasound transducer
349: inclined surface portion
352: observation window
354: illumination window
356: air/water supply nozzle
358: treatment tool outlet port
360: balloon
360A: balloon body
360B: annular locking part
361B: end part
370: elevator
390: balloon attachment groove
396: supply/discharge port
440: distal end part
460: balloon
460A: balloon body
460B: annular locking part
461B: toric part
462B: toric protrusion part
490: balloon attachment groove
540: distal end part
560: balloon
560A: balloon body
560B: annular locking part
561B: fitting shape part
590: balloon attachment groove

What is claimed is:

1. An ultrasonic endoscope comprising:
an ultrasound oscillator unit disposed in a distal end part of an insertion part; and
a balloon attachably and detachably attached to the distal end part to cover an outside surface of the ultrasound oscillator unit,
wherein the distal end part is provided on at least one of a distal end side or a proximal end side of the ultrasound oscillator unit and has a balloon attachment groove formed in an outer peripheral surface of the distal end part along a peripheral direction,
the balloon has a balloon body that has at least one end provided with an opening, and an annular locking part that is provided in the opening of the balloon body and is locked in the balloon attachment groove, and
the annular locking part has a detachment prevention shape for restraining detachment from the balloon attachment groove.

2. The ultrasonic endoscope according to claim 1,
wherein, in a cross-sectional shape in a case where the annular locking part is cut in an axial direction perpendicular to a peripheral direction of the annular locking part, the annular locking part has, as the detachment prevention shape, a shape in which a thickness on a balloon body side is thickest and the thickness is thinned toward an end portion on an opposite side to the balloon body.

3. The ultrasonic endoscope according to claim 1,
wherein the annular locking part has a toric part that is connected to the balloon body and is positioned on an outside surface of the distal end part, and a toric protrusion part that protrudes in an inward direction from the toric part and is fitted into the balloon attachment groove,
in a cross-sectional shape in a case where the annular locking part is cut in an axial direction perpendicular to a peripheral direction of the annular locking part, in a case where the axial direction is set as a width direction, the toric part is formed to be wider than the toric protrusion part, and
the detachment prevention shape includes the toric part and the toric protrusion part.

4. The ultrasonic endoscope according to claim 3,
wherein a relationship of a height of the toric protrusion part greater than a height of the toric part is satisfied in a case where a direction perpendicular to the axial direction is set as a height direction in the cross-sectional shape of the annular locking part.

5. The ultrasonic endoscope according to claim 3,
wherein the toric part has an internal extension part that extends from a protruding position of the toric protrusion part toward a balloon body side on the outer peripheral surface of the distal end part.

6. The ultrasonic endoscope according to claim 5,
wherein, in a case where the axial direction is set as the width direction in the cross-sectional shape of the annular locking part, and in a case where a width of the toric protrusion part is C, and a width of the internal extension part of the toric part is D₁, C/2 ≤ D₁ ≤ 2C is satisfied.

7. The ultrasonic endoscope according to claim 3,
wherein the toric part has an external extension part that extends from a protruding position of the toric protrusion part toward an opposite side to a balloon body side on the outer peripheral surface of the distal end part.

8. The ultrasonic endoscope according to claim 7,
wherein, in a case where the axial direction is set as the width direction in the cross-sectional shape of the annular locking part, and in a case where a width of the toric protrusion part is C, and a width of the external extension part of the toric part is D₂, C/2 ≤ D₂ ≤ 2C is satisfied.

9. The ultrasonic endoscope according to claim 1,
wherein, in a cross-sectional shape in a case where the annular locking part is cut in an axial direction perpendicular to a peripheral direction of the annular locking part, in a case where the axial direction is set as a width direction, the annular locking part has, as the detachment prevention shape, a fitting shape part in which an inside in a radial direction perpendicular to the axial direction is formed to be wider than an outside, and
the balloon attachment groove has a groove shape in which the fitting shape part is fitted into the groove in the width direction with no gap.

10. The ultrasonic endoscope according to claim 9,
wherein the fitting shape part has a shape that is widened to a balloon body side from the outside toward the inside in the radial direction.

11. The ultrasonic endoscope according to claim 1,
wherein the annular locking part is formed separately from the balloon body and is integrated with the balloon body.

12. The ultrasonic endoscope according to claim 11,
wherein the annular locking part is bonded to the balloon body by adhesion or welding.

13. The ultrasonic endoscope according to claim 11,
wherein the annular locking part is disposed on an inner surface side of the balloon body.

14. The ultrasonic endoscope according to claim 1,
wherein a material of the balloon body is silicone.

15. The ultrasonic endoscope according to claim 1,
wherein the distal end part has, as the balloon attachment groove, a first balloon attachment groove provided on the proximal end side of the ultrasound oscillator unit, and a second balloon attachment groove provided on the distal end side of the ultrasound oscillator unit, and
the balloon has, as the annular locking part, a first annular locking part that is locked in the first balloon attachment groove, and a second annular locking part that is locked in the second balloon attachment groove.

16. A balloon that is used in an ultrasonic endoscope including an ultrasound oscillator unit disposed in a distal end part of an insertion part and having a balloon attachment groove that is provided on at least one of a distal end side or a proximal end side of the ultrasound oscillator unit and formed in an outer peripheral surface of the distal end part along a peripheral direction, and is attachably and detachably attached to the distal end part to cover an outside surface of the ultrasound oscillator unit, the balloon comprising:
a balloon body that has at least one end provided with an opening; and
an annular locking part that is provided in the opening of the balloon body and is locked in the balloon attachment groove,
wherein the annular locking part has a detachment prevention shape for restraining detachment from the balloon attachment groove.
